(19) United States
(12) Patent Application Publication
(10) Pub. No.: US 2016/0058897 A1
(43) Pub. Date: Mar. 3, 2016

(54) KIT AND METHOD FOR QUICKLY PREPARING RADIO-ISOTOPE LABELED HUMAN SERUM ALBUMIN MICROSPHERES

(71) Applicant: Institute of Nuclear Energy Research Atomic Energy Council, Executive Yuan, TAOYUAN COUNTY (TW)

(72) Inventors: SU-JUNG CHEN, TAOYUAN COUNTY (TW); CHUNG-YEN LI, TAOYUAN COUNTY (TW); TE-WEI LEE, Taipei City (TW); CHIH-HSIEN CHANG, Hsinchu City (TW)

(21) Appl. No.: 14/508,243

(22) Filed: Oct. 7, 2014

(30) Foreign Application Priority Data
Sep. 2, 2014 (TW) 103130273

(51) Int. Cl. A61K 51/08; B65D 85/84; B65D 85/00; B65D 81/32; C07F 13/00; A61K 9/16

(52) U.S. Cl. CPC A61K 51/081; C07F 13/00; A61K 9/1682; B65D 85/70; B65D 81/32; B65D 85/84

(57) ABSTRACT

The present disclosure relates to a kit for preparing radio-isotope labeled human serum albumin (HSA) microspheres, which includes: a container (A), containing SnCl₂ dissolved in an aqueous acid solution; a container (B), containing an acidic substance as a tin salt stabilizer; a container (C), containing HSA microspheres to be labeled by a radio-isotope; and a container (D), containing a pH adjuster. According to the present kit for quickly preparing radio-isotope labeled HSA microspheres, HSA microspheres can be simply and quickly labeled by a radio-isotope at high labeling efficiency.

The present disclosure also relates to a method for quickly preparing radio-isotope labeled HSA microspheres by using the kit.

### TECHNICAL FIELD

The present disclosure relates to a kit for quickly preparing radio-isotope labeled human serum albumin (HSA) microspheres and a method for quickly preparing radio-isotope labeled HSA microspheres by using the kit, which can be applied to angiographic diagnosis and treatment of tumors by simply and quickly labeling a radio-isotope on HSA microspheres at high labeling efficiency.

### BACKGROUND

As a drug for radiotherapy of tumor in vivo, radio-isotope labeled microspheres have been widely used in tumor treatment, and the main treatment manner is directly guiding radioactive microspheres to a tumor site by using a duct. Since radioactive microspheres have a particle diameter greater than the microvascular diameter, the microvessels of the tumor are closured, so the supply of nutrient to the tumor is blocked, so necrosis of the tumor is accelerated. Moreover, the radioactive microspheres may also be concentrated at the tumor site to selectively increase the radioactivity to directly give damage to tumor cells, which can reduce the damage to other normal cells from the radioactive drug. In order to achieve therapeutic effect, many radio-isotopes such as yttrium-90 (⁹⁰Y), rhenium-188 (¹⁸⁸Re), rhenium-186 (¹⁸⁶Re), technetium-99m (^{99m}Tc) and holmium-166 (¹⁶⁶Ho) have been studied as nuclides for radiotherapy and are labeled on microspheres. Isotope rhenium-188 can release β-ray, has a maximum energy of 2.12 MeV and a half-life of 16.9 hr, and is suitable for being used in treatment; and at the same time, isotope rhenium-188 can release γ-ray of 155 KeV, and is suitable for being used in angiographic diagnosis. This nuclide is generated by a tungsten-188/rhenium-188 generator, which is convenient to use in hospital.

Microspheres synthesized with HSA microspheres as raw material are ideal radio-isotope supports, and have the following advantages: (1) being biodegradable and biocompatible: different from other microspheres synthesized with glass or plastics as raw material, the HSA microspheres is biodegradable and has no antigenicity, thus avoiding the security risk of permanently remained in human body; (2) being capable of performing labeling reaction in a high-temperature environment, thus facilitating radionuclide labeling; (3) having high drug stability after labeling; and (4) using microspheres in a specific size range, be capable of maintaining stable structure and configuration in a high-temperature environment, and being suitable for radionuclide labeling.

In 2005, Wunderlich set forth a method for labeling HSA microspheres with rhenium-188, where according to the labeling process, rhenium-188 is reduced by stannous chloride, and reduced rhenium-188 is adsorbed and precipitate on the surface of the microspheres (US20080219923 A1). Although this method significantly improves the labeling efficiency and reduces the amount of stannous chloride, the reaction needs to be performed in a water bath of about 90° C., and 90% labeling efficiency can be achieved after a reaction time of 45 to 70 min, while for synthesis of drugs labeled by a radio-isotope having a short half-life, the activity loss of the radio-isotope needs to be taken into consideration, so the long period of reaction time is not conducive to commercial applications. Moreover, it is found that the stability in serum is reduced with time, and after 48 hr at room temperature, the stability is reduced to 86% of the original value. Upon in vivo applications, due to the reduced stability, the efficacy is influenced, and free rhenium-188 on the surface of the microspheres may be distributed on other parts of the organism, thus causing risks of toxicity.

### SUMMARY

The present invention is completed in view of the above situation, and objectives of the present invention are to provide a kit and a method for simply and quickly preparing radio-isotope labeled HSA microspheres by labeling a radio-isotope on HSA microspheres at high labeling efficiency.

The present invention provides a kit for quickly preparing radio-isotope labeled HSA microspheres, which comprises: a container (A), containing SnCl₂ dissolved in aqueous acid solution; a container (B), containing a tin salt stabilizer used for radio-isotope stabilization; a container (C), containing HSA microspheres having a particle diameter in the range of 10 to 60 μm and to be labeled by a radio-isotope; a container (D), containing a pH adjuster for adjusting the pH value suitable for administration to human body such as a pH value in the range of 6 to 8 after bonding and a reduction reaction of the reagents in the containers (A) to (C) and the radio-isotope nuclide are completed.

According to the kit for quickly preparing radio-isotope labeled HSA microspheres of the present invention, the aqueous acid solution in the container (A) is at least one selected from aqueous hydrochloric acid, phosphoric acid and acetic acid solution.

According to the kit for quickly preparing radio-isotope labeled HSA microspheres of the present invention, the tin salt stabilizer in the container (B) is at least one selected from citric acid, oxalic acid, gallic acid, salicylic acid, tartaric acid, gluconic acid, ascorbic acid and benzoic acid.

According to the kit for preparing radio-isotope labeled HSA microspheres of the present invention, the particle diameter of the HSA microspheres in the container (C) is in the range of 10 to 60 μm, and the HSA microspheres may be HSA microspheres prepared by a conventional method and may also be commercially available HSA microspheres, for example, model ROTOP-HSA B20 purchased from Rotop Company (Germany), containing 2.5 mg HSA microspheres per dose and containing 300,000 to 500,000 microspheres having a particle diameter of about 10 to 30 μm, but not limited thereto, provided that HSA microspheres having a particle diameter in the range of 10 to 60 μm can be used.

According to the kit for preparing radio-isotope labeled HSA microspheres of the present invention, the pH adjuster in the container (D) is not particularly limited and may be a basic compound that can adjust the pH value of the solution finally obtained after mixing the reagents in containers (A) to (C) to the range described above, and includes, for example, NaOH, ammonia, Tris buffer, PBS buffer and phosphate, with an aqueous NaOH solution being preferred. The concentration and amount of the pH adjuster are also not particularly limited, provided that the pH value of the solution finally obtained after mixing the reagents in containers (A) to (C) is adjusted to the range described above.

According to the kit for preparing radio-isotope labeled HSA microspheres of the present invention, for the convenience in use, the kit dose may be designed to be a single dose after mixing, so that merely one kit is needed to complete mixing of required reagents upon each time of use. At this time, the contents of the reagents in the containers are respectively as follows, the mass of SnCl₂ in the container (A) is in the range of 2.5 mg to 10 mg, and preferably in the range of 3.5 mg to 4.5 mg, the mass of the tin salt stabilizer in the container (B) is in the range of 10 mg to 30 mg, and preferably in the range of 15 mg to 25 mg, and the mass of the HSA microspheres in the container (C) is in the range of 2.0 mg to 3.0 mg. According to the specification of the designed dose, the amount of the radio-isotope for labeling the HSA microspheres is 10 to 200 mCi/mL by specific activity.

The nuclide to be labeled by a radio-isotope by using the kit of the present invention may be rhenium-188 (¹⁸⁸Re), rhenium-186 (¹⁸⁶Re), technetium-99m (^{99m}Tc), and includes, for example, sodium perrhenate (¹⁸⁸ReO₄Na or ¹⁸⁶ReO₄Na) or sodium technetate (^{99m}TcO₄Na) (which can generate perrhenate radical (¹⁸⁸ReO₄⁻ or ¹⁸⁶ReO₄⁻) or technetate radical (^{99m}TcO₄⁻) in an aqueous solution through disassociation, which can be reduced into radio-isotope rhenium-188 (¹⁸⁸Re) or rhenium-186 (¹⁸⁶Re) or technetium-99m (^{99m}Tc) from a high oxidation number to a low oxidation number by SnCl₂.

A method for preparing radio-isotope labeled HSA microspheres, which is characterized by using the kit for labeling HSA microspheres with a radio-isotope comprising containers (A) to (D), and comprising steps of:

(1) respectively injecting a saline solution into a container (A) and a container (B) to form an aqueous solution, next, respectively injecting the solution in the container (A) and the solution in the container (B) to a container (C), and then, injecting a radio-isotope into the container (C) and fully mixed;

(2) placing the container (C) in the step (1) into a microwave reactor for a labeling reaction at a microwave power of 40 to 200 W for a reaction time of 1 to 10 min, and at the same time, reducing the radio-isotope from a high oxidation number to a low oxidation number, and bonding the radio-isotope on the HSA microspheres, to obtain radio-isotope labeled HSA microspheres,

where, according to the method for preparing radio-isotope labeled HSA microspheres of the present invention, the radio-isotope may be rhenium-188 (¹⁸⁸Re), rhenium-186 (¹⁸⁶Re) or technetium-99m (^{99m}Tc); and

(3) adding a pH adjuster in a container (D) to the radio-isotope labeled HSA microspheres obtained in Step (2) to adjust the pH value to a pH value in the range of 6 to 8.

According to the method for preparing radio-isotope labeled HSA microspheres of the present invention, since the radio-isotope easily decays with time, before starting the method of the present invention, elutriation of the radio-isotope nuclide is performed. A generator for manufacturing the radio-isotope nuclide is not particularly limited, but, for example, the ¹⁸⁸W/¹⁸⁸Re generator manufactured by the National Institute for Radioelement (IRE, Belgium) can be used for elutriation.

The method for preparing radio-isotope labeled HSA microspheres of the present invention has the following advantages: (1) being easy to operate and high labeling efficiency, so that the reaction time can be reduced, thus being suitable for a radio-isotope that decays with time, especially a radio-isotope having a short half-life, achieving the benefit of low cost, and increasing the useful life of radioactive drugs due to the reduced reaction time; (2) being applicable in treatment of hepatic artery embolization microspheres due to the particle diameter in the range of 10 to 60 μm and ingredients being easily degraded (3) having good drug stability; and (4) having double efficacies of radiodiagnosis and cancer treatment.

### BRIEF DESCRIPTION OF DRAWINGS

FIG. 1 is a flowchart of implementation of a method for preparing radio-isotope labeled HSA microspheres of the present invention; and

FIG. 2 is chart of analysis results of stability over time of ¹⁸⁸Re-HSA microspheres according to Example 3.

### DETAILED DESCRIPTION OF DISCLOSED EXAMPLES

In the following, the present invention is further described with a Synthetic Example and Examples, but the Synthetic Example and Examples are merely used to illustrate the present invention, but not intended to limit the scope of the present invention. For example, in the following Examples, merely rhenium-188radio-isotope is used as an example for illustration, but the present invention is not limited thereto, and rhenium-186 (¹⁸⁶Re) or technetium-99m (^{99m}Tc) may also be used as the radio-isotope.

### Synthesis Example Synthesis of HSA Microspheres

Step 1: To a 1,000 mL breaker, 800 mL olive oil was added, and then the breaker was placed on a temperature-controlled heater equipped with a magnet heating mixer, and heated to a temperature of 60° C. for pre-heating for at least 30 min at a rotation rate of 520 rpm. In the temperature-controlled heater, a micro-motor drives a high temperature-resistant powerful magnet to rotate and generate a rotating magnetic field, so that stirrers in a container are driven to rotate, and at the same time, the solution in the container is heated simultaneously, so as to allow the solution to be fully mixed and reacted.

Step 2: HSA (purchased from Sigma Company) was placed in 5 mL water to formulate an HSA solution having a concentration of 20% (w/v), and the HSA solution was drawn by a pump and added dropwise into the breaker at an initial rate of 4 mL/min, and then the rate was gradually increased to 9 mL/min till addition was finished, and then the mixture was stirred for 3 min at a rotation rate of 520 rpm.

Step 3: The rotation rate of the temperature-controlled heater was adjusted to 240 rpm and the reaction was performed for 30 min, then the reaction temperature was adjusted to 110° C., and the reaction was performed for 30 min at a rotation rate of 50 rpm.

Step 4: Olive oil was removed after reaction by a 20 m filter, oil molecules on the surface of the HSA microspheres was washed by acetone, and the resulting product was dried for a certain period of time at 40° C., and HSA microspheres having a particle diameter in the range of 10 to 60 m were sieved out by a 60 m screen and a 10 m screen.

### Example 1

### Preparation of HSA Microsphere Kit

4 mg SnCl₂ and 400 uL 0.1N HCl were fully mixed and dissolved, nitrogen was charged into the container for about 1 min, after freeze-drying, and the container cap was sealed with plastic cork and aluminum cap, to obtain a container (A). Additionally, 20 mg citric acid was added into a container (B), nitrogen was charged into the container for about 1 min, and the container cap was sealed with plastic cork and aluminum cap. Additionally, 2.5 mg HSA microspheres prepared in Synthetic Example was placed in a container (C), nitrogen was charged into the container for about 1 min, and the container cap was sealed with plastic cork and aluminum cap. Additionally, 1 mL 1N NaOH was placed in a container (D), and the container cap was sealed with plastic cork and aluminum cap.

### Example 2

### Preparation and Analysis of Rhenium-188 Labeled HSA Microspheres

1. 400 uL saline solution was added to a container (A) and fully mixed, to obtain a mixture (A). 400 uL saline solution was added to a container (B) and fully mixed, to obtain a mixture (B).

2. The mixture (A) and the mixture (B) obtained in Step 1 were extracted and injected into a container (C), and 600 μL radio-isotope rhenium-188 solution was injected to the container (C), where the activity of rhenium-188 was 25 mCi/mL.

3. The mixture obtained in Step 2 was transferred into a microwave reactor for a labeling reaction for 3 min at a microwave power of 90 W, to obtain a rhenium-188 labeled HSA microspheres (referred to as ¹⁸⁸Re-HSA microspheres thereafter)-containing mixture.

4. Next, 300 μL solution in the container (D) was drawn and injected into the ¹⁸⁸Re-HSA microspheres-containing mixture obtained in Step 3, to adjust the pH value to be 7.0.

5. Then, labeling efficiency measurement and particle diameter analysis were performed, the ¹⁸⁸Re-HSA microspheres-containing mixture obtained in Step 4 was centrifuged for 5 min at 13,000 rpm, and the supernatant and precipitate were respectively taken for radioactivity measurement, and the labeling efficiency of the ¹⁸⁸Re-HSA microspheres was calculated to be 97% according to the following formula labeling efficiency.

Labeling efficiency (%)=[(precipitate activity)/(supernatant activity+precipitate activity)]×100%.

The particle diameter of the ¹⁸⁸Re-HSA microspheres was measured by using a micron particle size analyzer, and the average particle diameter of the ¹⁸⁸Re-HSA microspheres was measured to be about 24 μm.

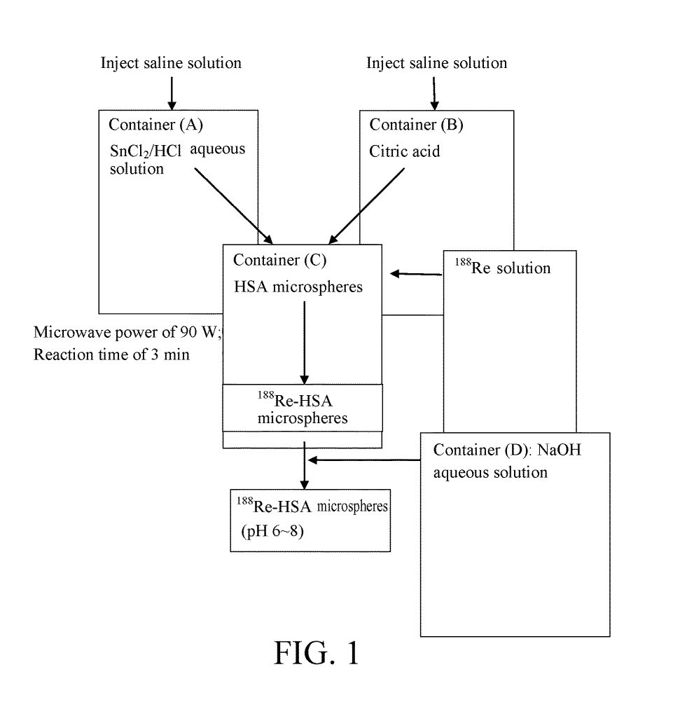

The flowchart of Steps 1 to 4 is shown in FIG. 1.

### Example 3

### Analysis of Stability Over Time of ¹⁸⁸Re-HSA Microspheres

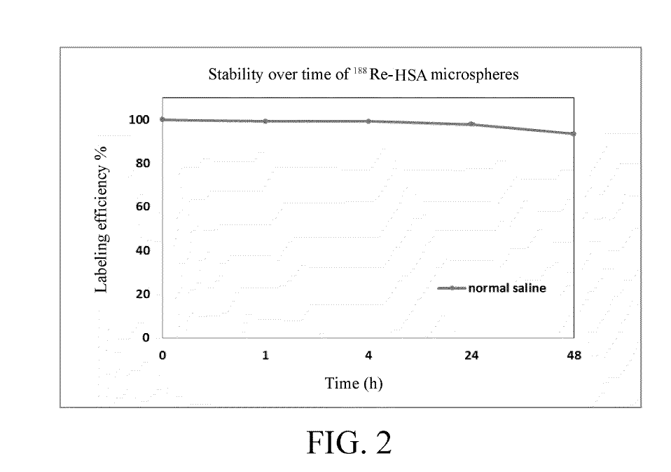

At room temperature, the ¹⁸⁸Re-HSA microspheres-containing mixture obtained in Example 2 was placed in 3000 μL saline solution, 500 μL mixture was drawn respectively at 1 hr, 4 hr, 24 hr and 48 hr for labeling efficiency measurement by the method in Step 5 of Example 2. It can be known from the test results that, the ¹⁸⁸Re-HSA microspheres still has a labeling efficiency greater than 90% after 48 hr in the saline solution, indicating that the ¹⁸⁸Re-HSA microspheres prepared by the method of the present invention is considerably stable. The results are shown in FIG. 2 and Table 1.

TABLE 1
Results of stability over time

| | Microspheres bonded Radioactivity, |
|---|---|
| Time (hr) | Mean ± SD in saline solution, 25° C. |
| 1 | 99.30 ± 0.16 |
| 4 | 99.20 ± 0.22 |
| 24 | 97.93 ± 0.67 |
| 48 | 93.53 ± 0.70 |

Mean ± Standard deviation (SD), n = 3

It can be known from the above that, the method for quickly preparing radio-isotope labeled HSA microspheres of the present invention has the following advantages: (1) being easy to operate and high labeling efficiency, so that the reaction time can be reduced, thus being suitable for a radio-isotope that decays with time, especially a radio-isotope having a short half-life, achieving the benefit of low cost, and increasing the useful life of radioactive drugs due to the reduced reaction time; (2) being applicable in treatment of hepatic artery embolization microspheres due to the particle diameter in the range of 10 to 60 μm and ingredients being easily degraded (3) having good drug stability; and (4) having double efficacies of radiodiagnosis and cancer treatment.

What is claimed is:

1. A kit for quickly preparing radio-isotope labeled human serum albumin (HSA) microspheres, comprising: a container (A), containing SnCl₂ dissolved in an aqueous acid solution; a container (B), containing a tin salt stabilizer; a container (C), containing HSA microspheres to be labeled by an isotope; and a container (D), containing a pH adjuster.

2. The kit according to claim 1, wherein the aqueous acid solution in the container (A) is at least one of hydrochloric acid, phosphoric acid and acetic acid.

3. The kit according to claim 1, wherein the tin salt stabilizer in the container (B) is at least one selected from citric acid, oxalic acid, gallic acid, salicylic acid, tartaric acid, gluconic acid, ascorbic acid and benzoic acid.

4. The kit according to claim 1, wherein the particle diameter of the HSA microspheres in the container (C) is in the range of 10 to 60 μm.

5. The kit according to claim 1, wherein as for a single dose after mixing as designed, the contents of the reagents in the containers are respectively as follows, the mass of SnCl₂ in the container (A) is in the range of 2.5 mg to 10 mg, the mass of the tin salt stabilizer in the container (B) is in the range of 10 mg to 30 mg, and the mass of the HSA microspheres in the container (C) is in the range of 2.0 mg to 3.0 mg.

6. The kit according to claim 1, wherein the pH adjuster in the container (D) is at least one selected from NaOH, ammonia, Tris buffer, PBS buffer and phosphate.

7. A method for quickly preparing radio-isotope labeled human serum albumin (HSA) microspheres, which is characterized by using the kit according to claim 1, and comprising steps of:
(1) respectively injecting a saline solution into a container (A) and a container (B), next, respectively injecting the solution in the container (A) and the solution in the container (B) into a container (C), and then, injecting a radionuclide solution into the container (C);
(2) placing the container (C) in the step (1) into a microwave reactor for a labeling reaction at a specific microwave power for an appropriate reaction time; and
(3) adding the reactant to a container (D) to adjust to an appropriate pH value, to obtain radio-isotope labeled HSA microspheres.

8. The method according to claim 7, wherein the radionuclide in the radionuclide solution in Step (1) is at least one of rhenium-188 (¹⁸⁸Re) or rhenium-186 (¹⁸⁶Re) and technetium-99m (^{99m}Tc).

9. The method according to claim 7, wherein the specific microwave power in Step (2) is 40 to 200 W.

10. The method according to claim 7, wherein the appropriate reaction time in Step (2) is 1 to 10 min.

11. The method according to claim 7, wherein the appropriate pH value in Step (3) is in the range of 6 to 8.
